(19) United States
(12) Patent Application Publication
(10) Pub. No.: US 2026/0357204 A1
(43) Pub. Date: Nov. 12, 2026

(54) Compositions and Methods for Milk-based Mammary Epithelial Cell Platforms for Assessment of Breast Tissue Health

(71) Applicant: University of Houston System, Houston, TX (US)

(72) Inventors: Tasneem Bawa-Khalfe, Houston, TX (US); Samaneh Karami, Houston, TX (US)

(21) Appl. No.: 19/674,008

(22) Filed: May 11, 2026

Related U.S. Application Data
(60) Provisional application No. 63/803,590, filed on May 10, 2025.

(51) Int. Cl. C12N 5/071; C12Q 1/6886

(52) U.S. Cl. CPC C12N 5/0631; C12Q 1/6886; C12Q 2600/158

(57) ABSTRACT

Provided herein are breast-derived cellular compositions comprising mammary epithelial cells obtained from human milk samples, mammary organoids produced therefrom, and methods of making and using the same. In certain embodiments, a human milk sample is processed to isolate or enrich mammary epithelial cells, which may be cultured under organoid-forming conditions, including in a three-dimensional matrix, to generate a mammary organoid. The composition or mammary organoid may be evaluated for one or more biomarkers associated with breast cancer. Further provided are methods of assessing a condition of breast tissue in a subject by obtaining a human milk sample, isolating or enriching mammary epithelial cells, culturing the cells to produce a mammary organoid or cultured mammary epithelial cell preparation, measuring a level or pattern of one or more biomarkers associated with a breast-associated abnormality, and comparing the measured level or pattern to a reference profile to detect the breast-associated abnormality, assess risk of the breast-associated abnormality, monitor disease, determine prognosis, or evaluate treatment response.

### CROSS-REFERENCE TO RELATED APPLICATIONS

This application claims the benefit of and priority to U.S. Provisional Application No. 63/803,590, filed on May 10, 2025, the disclosure of which is incorporated by reference herein in its entirety.

### GOVERNMENT SUPPORT

This invention was made with government support under grant number: U54MD015946 awarded by the National Institute on Minority Health and Health Disparities of the National Institutes of Health and R01CA256543 from the National Cancer Institute of the National Institutes of Health. The government has certain rights in the invention.

### TECHNICAL FIELD

The present disclosure relates generally to the fields of cancer diagnostics, liquid biopsy technologies, cellular analysis, and breast health assessment. More specifically, the disclosure relates to the use of human milk as a breast-specific liquid biopsy source for obtaining samples. These methods and platforms may be used as non-invasive tools for evaluation of breast tissue health, breast cancer risk assessment, early disease detection, disease monitoring, and/or evaluation of therapeutic response and treatment efficacy.

### BACKGROUND

Early detection of breast cancer (BCa) significantly improves survival rates, yet current screening methods have limitations that hinder timely diagnosis, especially in younger, postpartum, and lactating women. Mammography, the most widely used screening tool, has reduced sensitivity in dense breast tissue, leading to false negatives, equivocal findings, and delayed detection. These limitations are particularly relevant in women of childbearing age, who frequently present with increased breast density and physiologic breast remodeling associated with pregnancy and lactation. In addition, existing diagnostic approaches may be invasive, costly, and inaccessible, particularly for women in underserved communities or those who are pregnant or lactating and therefore often excluded from routine screening paradigms. Imaging-based modalities may also fail to capture early molecular and cellular alterations that precede formation of clinically detectable lesions. Accordingly, there remains a need for a non-invasive, breast-specific platform capable of obtaining viable mammary-derived cells for molecular, cellular, and functional analysis during this high-risk window.

### SUMMARY

Provided herein are breast-derived cellular compositions comprising mammary epithelial cells obtained from a human milk sample, wherein the mammary epithelial cells are capable of being cultured under organoid-forming conditions to produce a mammary organoid. In certain embodiments, the mammary epithelial cells are disposed in a three-dimensional matrix, including collagen, laminin, heparan sulfate proteoglycans, entactin/nidogen, growth factors, or a combination thereof. In certain embodiments, the resulting mammary organoid exhibits secretory differentiation, branching morphology, milk secretion, or a combination thereof. In certain embodiments, the composition or organoid is evaluated for one or more biomarkers associated with proliferation, epithelial differentiation, stemness, oncogenic transformation, or epithelial-to-mesenchymal transition.

Further provided are methods of producing a breast-derived cellular composition or a mammary organoid by obtaining a human milk sample, processing the human milk sample to provide mammary epithelial cells, and culturing the mammary epithelial cells under conditions sufficient to generate the breast-derived cellular composition or the mammary organoid. In certain embodiments, the processing includes washing, centrifuging, enriching, sorting, thawing, or a combination thereof, and the culturing includes culturing the mammary epithelial cells in a three-dimensional matrix. In certain embodiments, the mammary organoid is exposed to one or more pregnancy-associated hormones, lactation-associated hormones, or a combination thereof, and optionally subjected to hormone withdrawal to model an involution or postpartum state. Also provided are methods of assessing a condition of breast tissue in a subject by obtaining a human milk sample from the subject, isolating or enriching mammary epithelial cells from the human milk sample, culturing the mammary epithelial cells to produce a mammary organoid or cultured mammary epithelial cell preparation, measuring a level or pattern of one or more biomarkers associated with a breast-associated abnormality, and comparing the measured level or pattern to a reference profile to identify a reference profile to detect the breast-associated abnormality, assess risk of the breast-associated abnormality, monitor disease, determine prognosis, or evaluate treatment response. In certain embodiments, the breast-associated abnormality is breast cancer. In certain embodiments, the breast cancer can be postpartum breast cancer, the measuring includes digital droplet polymerase chain reaction or microscopy-based image analysis, and the comparing includes comparison to a non-cancer control, a postpartum control, a prior sample from the subject, or a control morphology profile, including in longitudinal analyses using samples obtained at different times from the same subject. In certain embodiments, breast-associated abnormalities include benign proliferative changes, atypical ductal or lobular hyperplasia, ductal carcinoma in situ (DCIS), lobular carcinoma in situ (LCIS), hormone receptor-positive or -negative lesions, HER2-amplified lesions, high-proliferation phenotypes marked by elevated biomarkers, and invasive ductal or lobular carcinomas.

### BRIEF DESCRIPTION OF THE DRAWINGS

The present disclosure can be better understood by referring to the following drawings. These drawings illustrate the principles of the disclosure and no limitation of the scope of the disclosure is thereby intended.

FIGS. 1A and 1B illustrate representative microscopy images of cultured epithelial cells maintained under control conditions and under hormone-based treatment conditions over multiple time points (day 6, day 12, and day 18). The images demonstrate visually observable differences in cellular morphology between cells cultured in the absence of hormone and cells cultured in the presence of hormone and the withdrawal of hormone, including prolactin, progesterone, and a lactation cocktail.

FIG. 2A-2H illustrate a series of microscopy images of nulliparous mice organoids under control conditions and under presence of hormone at various time points. Each panel provides a visual representation of cellular morphology corresponding to a distinct condition or experimental variable. FIG. 2I is a graphical representation of the mammary organoid size of the nulliparous mice organoids as measured by Image J analysis. FIG. 3A-3H illustrate a series of microscopy images of parous mice organoids under control conditions and under presence of hormone at various time points. Each panel provides a visual representation of cellular morphology corresponding to a distinct condition or experimental variable. FIG. 3I is a graphical representation of the mammary organoid size of the parous mice organoids as measured by Image J analysis.

FIG. 4A is an illustration of a method of a method of generating the organoid model from human milk-derived cells. FIG. 4B in the left and middle panels present bright-field microscopy images of cultured cells before and following hormone treatment. FIG. 4B in the right panel presents fluorescence microscopy image of cultured cells following hormone treatment, showing nuclei stained with DAPI. FIG. 4C illustrates a fluorescence microscopy image of cultured cells under control conditions and following hormone treatment, showing DAPI nuclear staining.

FIG. 5A-5D demonstrate characterization and viability analysis of human milk-derived cells isolated from fresh and frozen milk samples. FIGS. 5A and 5B illustrate representative flow cytometry analyses (using specific epithelial, progenitor, antibodies) to identify and distinguish epithelial-associated cell populations relative to control samples. FIG. 5C demonstrates the relative distribution of these cell populations within human milk-derived cellular preparations, confirming the presence of viable mammary epithelial-associated populations suitable for downstream culture and organoid generation. FIG. 5D demonstrates comparative viability analysis of human milk-derived cells isolated from fresh versus frozen samples. FIG. 5E is a graphical representation of the expression of CLDN, K18, K5, KI67, CDH1, c-Myc, DHFR, STAT3, FN1, and VIM in five HM-derived 3D organoids.

### DETAILED DESCRIPTION

All parous women are under a 10-30% increased risk of developing postpartum breast cancer (PPBC) for at least a decade after childbirth. Postpartum women with BCa at age of ≤35-45 years, have an increased risk for metastasis and death, with nearly twice the mortality in comparison to older postmenopausal women diagnosed with BCa. Older age at pregnancy, higher parity number, and hereditary predisposing mutations (i.e. BRCA) are considered risk factors in onset of PPBC and/or PPBC-associated mortality. PPBC, like late-onset BCa, exhibits estrogen receptor positive (ER+) and negative (ER−) subtypes but unlike late-onset BCa, PPBC susceptibility is linked to involution-driven restructuring of the mammary gland. Thus, investigation on the underlying biological mechanisms driving the differentiation of breast tissue after involution in the postpartum years and correlation with disparities in PPBC is crucial for identifying women at-risk.

The absence of suitable models for assessing postpartum breast cancer among populations having defined risk factors poses a significant challenge. Studies show that human milk contains viable cells belonging to the epithelial luminal progenitor lineage. Accordingly, human milk-derived cells offer a unique opportunity to gain deeper insight into functional mammary gland changes during lactation and the postpartum period. These cells also have the potential to serve as early indicators of future breast cancer development, thereby contributing to the creation of a preclinical tool for early breast cancer detection. Because organoids recapitulate organ-of-origin architecture and retain molecular features, postpartum mammary organoids derived from human milk cells can provide a useful platform for elucidating mechanisms of postpartum breast cancer development across different parity states, ages at childbirth, and pregnancy lengths.

Nevertheless, there is currently a lack of an appropriate human three-dimensional organoid model that faithfully replicates key aspects of lactation, involution, and the postpartum phase. Existing systems fail to capture patient-specific biology during this high-risk window. Accordingly, provided herein is a cost-effective and non-invasive approach utilizing human milk-derived cells to generate organoid systems that enable assessment of postpartum breast cancer susceptibility and evaluation of prognosis.

In the following description, numerous details are set forth in order to provide a thorough understanding of the various embodiments. In other instances, well-known processes, devices, and systems may not been described in particular detail in order not to unnecessarily obscure the various embodiments. Additionally, illustrations of the various embodiments may omit certain features or details in order to not obscure the various embodiments. In the detailed description, reference is made to the accompanying drawings that form a part of this disclosure. Other embodiments may be utilized, and logical changes may be made without departing from the scope of this disclosure. The description may use the phrases “in some embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous.

Embodiments disclosed herein include breast-derived cellular compositions comprising mammary epithelial cells isolated from a human milk sample. Further embodiments include methods of assessing biomarkers associated with breast cancer susceptibility, detecting breast cancer, including postpartum breast cancer, and evaluating breast cancer risk using such compositions or mammary organoids produced therefrom. In some embodiments, the method includes isolating and culturing mammary epithelial cells from a human milk sample within a three-dimensional matrix under conditions sufficient to promote organoid formation. In certain embodiments, the mammary organoids are evaluated for one or more biomarkers associated with proliferation, epithelial differentiation, stemness, oncogenic transformation, or epithelial-to-mesenchymal transition. Specific biomarkers include, but are not limited to, Ki67, keratin 5, keratin 18, E-cadherin, claudin, STAT3, vimentin, dihydrofolate reductase, c-Myc, fibronectin, and combinations thereof. In certain embodiments, the mammary epithelial cells are cultured within a matrix to produce a functional mammary organoid exhibiting secretory differentiation capacity and the ability to produce milk in vitro.

In certain embodiments, the disclosed systems and methods provide a diagnostic and research platform utilizing human milk as a breast-specific, non-invasive liquid biopsy source for assessment of breast tissue condition, risk of developing breast abnormalities, and early disease detection. Mammary epithelial cells isolated from human milk may be cultured to generate mammary organoids that are analyzed for biomarkers and gene-expression profiles associated with susceptibility to breast abnormalities, disease progression, and therapeutic responses or lack thereof. In an embodiment, the breast abnormality is postpartum breast cancer (PPBC). Further embodiments may be described and disclosed.

In certain embodiments, a human milk sample obtained from a subject is processed to isolate or enrich mammary epithelial cells, and the mammary epithelial cells are cultured as a two-dimensional cultured mammary epithelial cell preparation or under organoid-forming conditions in a three-dimensional matrix to generate a mammary organoid for analysis. In some embodiments, the cultured cells or mammary organoid are exposed to one or more pregnancy-associated hormones, lactation-associated hormones, or combinations thereof, and optionally subjected to hormone withdrawal to model a postpartum or involution state. In further embodiments, the cultured cells or mammary organoid are analyzed for one or more biomarkers associated with proliferation, epithelial differentiation, stemness, oncogenic transformation, epithelial-to-mesenchymal transition, or breast cancer, including by digital droplet polymerase chain reaction, microscopy-based image analysis, or combinations thereof, and the resulting measurements are compared with a reference profile, a control morphology profile, a postpartum control, a parity-matched control, or a prior sample from the same subject to assess breast tissue condition, detect a breast-associated abnormality, assess risk, monitor disease progression, determine prognosis, or evaluate a treatment response.

In certain embodiments, the breast-associated abnormality includes a postpartum breast tissue abnormality, including an abnormality associated with lactation, involution, postpartum remodeling, atypical epithelial proliferation, premalignant change, or postpartum breast cancer susceptibility. In certain embodiments, breast-associated abnormalities include benign proliferative changes, atypical ductal or lobular hyperplasia, ductal carcinoma in situ (DCIS), lobular carcinoma in situ (LCIS), hormone receptor-positive or -negative lesions, HER2-amplified lesions, high-proliferation phenotypes marked by elevated biomarkers, and invasive ductal or lobular carcinomas. In some embodiments, a measured biomarker level, biomarker pattern, or morphology feature obtained from a cultured mammary epithelial cell preparation or mammary organoid is compared with a reference profile obtained from a healthy control, a postpartum control, a parity-matched control, an age-matched control, or a prior sample from the same subject to identify deviation from an expected postpartum or lactation-associated state.

In certain embodiments, measuring the one or more biomarkers includes digital droplet polymerase chain reaction performed on RNA, complementary DNA, or other nucleic acid material obtained from the mammary epithelial cells, the cultured mammary epithelial cell preparation, or the mammary organoid. In some embodiments, the digital droplet polymerase chain reaction is performed on a gene panel comprising at least two biomarkers selected from Ki67, keratin 5, keratin 18, E-cadherin, claudin, STAT3, vimentin, dihydrofolate reductase, c-Myc, fibronectin, and combinations thereof, and the resulting copy number, expression level, or fold-change is compared with a reference profile to identify a breast-associated abnormality, assess risk, or monitor progression.

In certain embodiments, measuring the one or more biomarkers additionally or alternatively includes microscopy-based image analysis of the mammary organoid or the cultured mammary epithelial cell preparation. In some embodiments, the image analysis quantifies one or more morphology features selected from organoid size, area, branching, circularity, lumen formation, lumen diameter, wall thickness, polarity, cellular organization, and combinations thereof. In further embodiments, the quantified morphology is compared with a control morphology profile to identify postpartum remodeling, involution-associated regression, abnormal epithelial organization, treatment response, or disease progression.

In certain embodiments, the method is performed using a first human milk sample and a second human milk sample obtained from the same subject at different times, and optionally one or more additional samples obtained over time. In some embodiments, a biomarker level, biomarker pattern, transcript copy number, or morphology feature from the first sample is compared with a corresponding biomarker level, biomarker pattern, transcript copy number, or morphology feature from the second sample to monitor treatment response, evaluate postpartum remodeling over time, detect disease progression, assess recurrence risk, or determine whether the subject is transitioning toward or away from a breast-associated abnormality.

Human milk-derived mammary epithelial cells are viable and can grow in three-dimensional culture systems. Additionally, these cells may include estrogen receptor-positive and estrogen receptor-negative populations and therefore represent a rich source of normal breast-derived cells. In certain embodiments, the disclosed system enables generation of a postpartum mammary gland model derived from a human milk sample. This model can be used as a platform to study epidemiological and biological factors, including parity-related factors, associated with breast cancer initiation and postpartum breast cancer development.

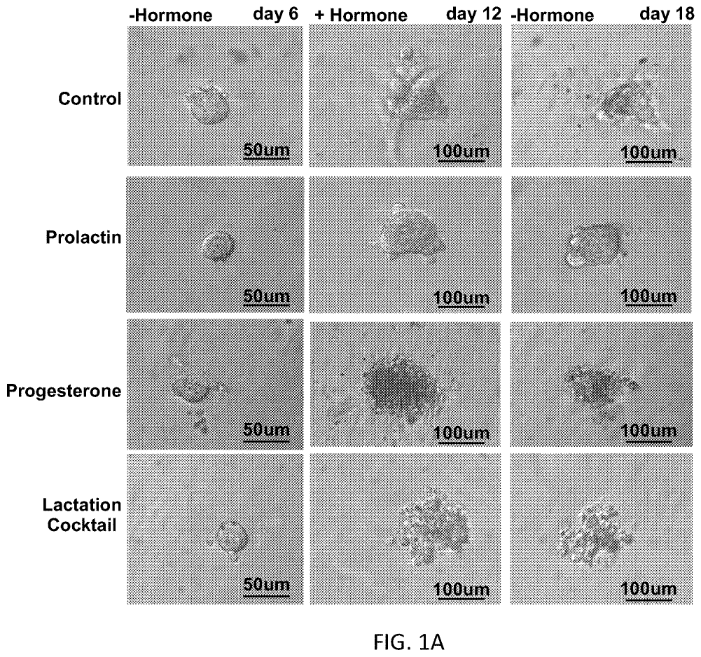

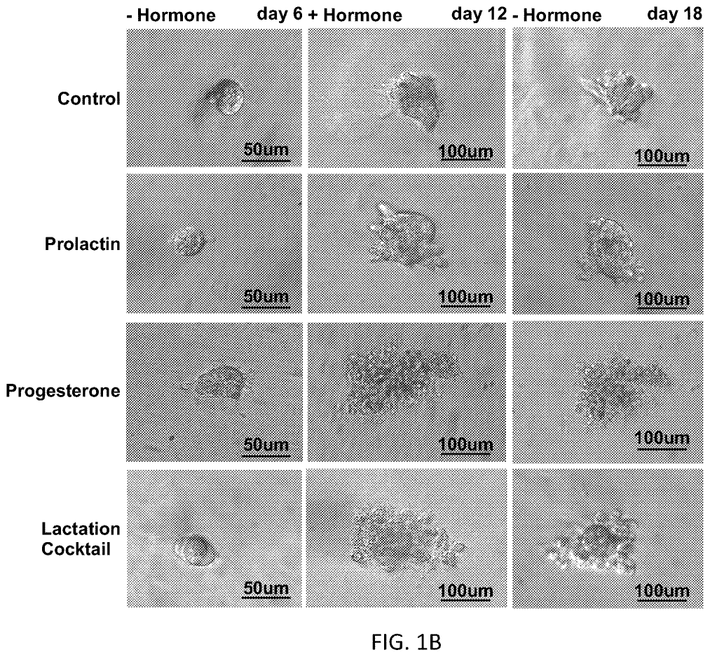

An embodiment includes a postpartum mammary gland model. FIGS. 1A and 1B illustrate representative microscopy images of cultured epithelial cells maintained under control conditions and under hormone-based treatment conditions over multiple time points (day 6, day 12, and day 18). The images demonstrate visually observable differences in cellular morphology between cells cultured in the absence of hormone and the presence of hormone and the withdrawal of hormone, including prolactin, progesterone, and a lactation cocktail. Under control conditions, organoids exhibited minimal branching and limited morphological changes. In contrast, hormone-treated organoids showed increased organoid expansion and branching morphology, hormones were withdrawn on day 12, and organoids imaged (same day, day 12). Organoids were cultured for an additional 6 days in the absence of hormone (day 18) and imaged on day 18 to evaluate involution-associated regression, specifically, branching and organoid size. At day 18, following hormone withdrawal, nulliparous organoids exhibit marked reduction in organoid size, branching, and overall structural organization, indicative of physiologically relevant involution-associated regression. In contrast, parous organoids (derived from mouse mammary glands of dams having undergone approximately 2-3 pregnancies) exhibit persistence of enlarged and highly branched structures with limited regression following hormone withdrawal, indicating reduced and defective postpartum remodeling and involution dynamics relative to nulliparous organoids. These parity-associated differences suggest that multiparity alters mammary gland remodeling responses during the postpartum phase and may contribute to increased susceptibility to postpartum breast cancer (PPBC). Consistently, the disclosed organoid systems recapitulate physiologically relevant postpartum and involution-associated changes and provide functional platforms for evaluation of PPBC risk, parity-associated breast cancer susceptibility, hormone responsiveness, and breast cancer-associated remodeling mechanisms. These embodiments are directed to methods of culturing epithelial cells, exposing the cells to one or more hormones, and maintaining the cells for a defined duration sufficient to induce a phenotypic change, wherein the change is detectable by optical microscopy.

In embodiments of this model, the organoids undergo a pregnancy/lactation process and then transition to an involution phase, thereby allowing study of factors that contribute to transformation of healthy luminal cells and postpartum breast cancer development.

Pregnancy/lactation hormone-treated nulliparous organoids undergo a notable morphological transformation characterized by distinct secretory branching patterns, similar to parous hormone-treated organoids, as compared to corresponding untreated control organoids. In certain embodiments, treatment with a combination pregnancy/lactation hormone cocktail demonstrates greater efficacy in inducing secretory differentiation, branching morphology, or functional maturation than treatment with individual hormones alone. In addition, when subjected to hormone withdrawal, nulliparous organoids exhibit postpartum morphology characterized by a reduction in size and branching. In certain embodiments, an exemplary lactation hormone cocktail contains one or more of prolactin, progesterone, hydrocortisone, oxytocin, fibroblast growth factor, and epidermal growth factor. A sample lactation hormone cocktail includes: 120 nm/ml Prolactin, 10 nm/ml Progesterone, Hydrocortisone, 10 nm/ml Oxytocin, 2.5 nm/ml FGF, and 10 ng/ml EGF.

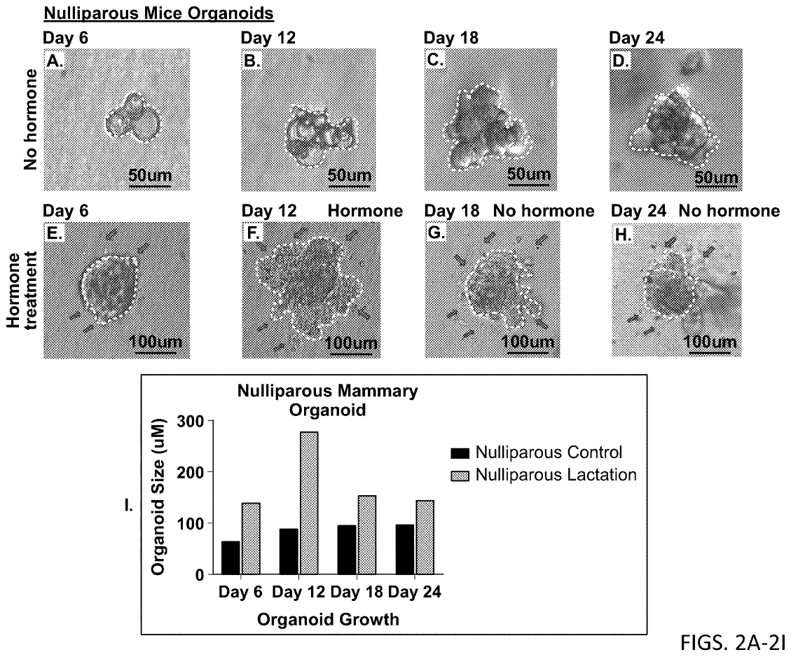

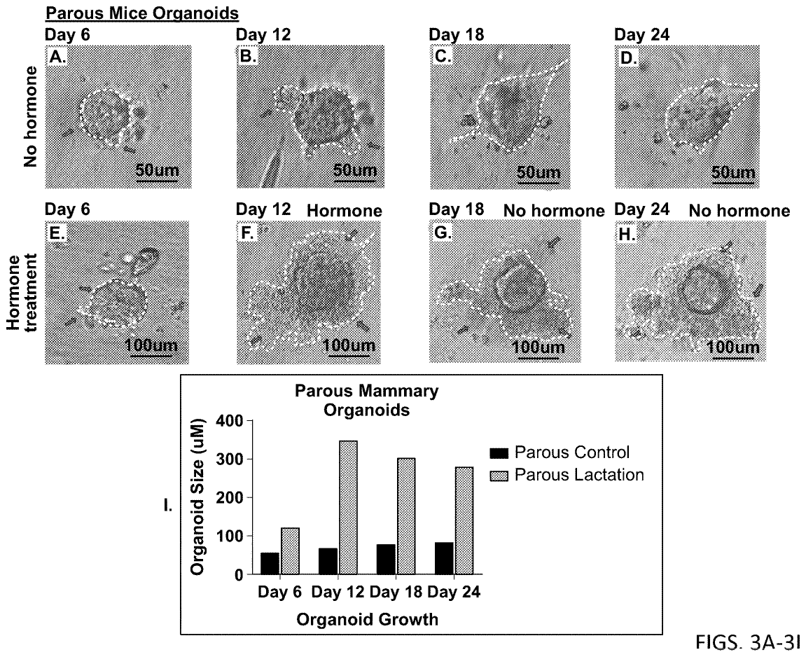

In contrast, postpartum organoid models derived from parous mammary gland (dams with 2-3 pregnancies), did not display substantial changes in branching when subjected to hormone deprivation (FIGS. 2G-2H). Organoid morphological analysis may be performed using image-analysis software (such as ImageJ software) to quantify organoid expansion, regression, structural remodeling, or other three-dimensional morphological characteristics. These data indicate that the postpartum organoid model mimics morphological aspects of the mammary gland in an in vivo-like environment and captures parity-associated biological transformation/changes.

FIG. 2A-2H illustrate a series of microscopy images of nulliparous mice organoids under control conditions and under presence of hormone at various time points. Each panel provides a visual representation of cellular morphology corresponding to a distinct condition or experimental variable. Under control conditions (absence of hormone), cultured organoids exhibited minimal branching, expansion, and morphological remodeling. In contrast, organoids treated with lactation hormone cocktail of prolactin, progesterone, hydrocortisone, oxytocin, FGF, and EGF demonstrated increased organoid expansion, branching morphogenesis, and secretory-like morphology. In certain embodiments, hormone-treated organoids were imaged on day 12 prior to hormone withdrawal. Hormone withdrawal was subsequently initiated on day 12 to mimic postpartum involution conditions. Microscopy followed on day 18 with organoids showing involution-associated regression, including reduced branching and organoid size following hormone withdrawal. FIG. 2I is a graphical representation of the mammary organoid size of the nulliparous mice organoids as measured by Image J analysis. FIG. 3A-3H illustrate a series of microscopy images of parous mice organoids under control conditions and under presence of hormone at various time points. Each panel provides a visual representation of cellular morphology corresponding to a distinct condition or experimental variable. FIG. 3I is a graphical representation of the mammary organoid size of the parous mice organoids as measured by Image J analysis. These embodiments are directed to methods of comparing cellular states across different culture conditions, including treated and untreated samples, and to evaluating structural features of cultured cells by imaging. FIGS. 2I and 3I demonstrate stage-specific development and remodeling of mammary organoids generated from nulliparous and parous mammary epithelial cells (MECs). At day 6 in the absence of hormone, both nulliparous and parous organoids exhibit similar baseline morphology with comparable no branching and organoid size. Following lactation-associated hormone treatment, both nulliparous and parous organoids undergo substantial morphological changes, including increased organoid size, expansion, and branching morphogenesis by day 12, indicating hormone-responsive growth, secretory-related differentiation, and development of a lactation organoid model. Following hormone withdrawal, nulliparous organoids exhibit reduction in organoid size and branching at day 18, indicative of involution-associated regression. In contrast, parous organoids (derived from mouse mammary glands of dams subject to 2-3 pregnancies) exhibit reduced and defective remodeling and involution-associated regression, with persistence of enlarged and branched structures following hormone withdrawal, consistent with multiparity-associated alterations in postpartum remodeling dynamics. Consistently, the disclosed organoid systems recapitulate physiologically relevant postpartum and involution-associated changes and provide functional platforms for evaluation of postpartum breast cancer (PPBC) risk, parity-associated breast cancer susceptibility, hormone responsiveness, and breast cancer-associated remodeling mechanisms.

In an embodiment, viable cells are collected non-invasively and successfully isolated from human milk donor samples under an approved institutional review board protocol. Cell viability may be assessed using Trypan Blue exclusion, flow cytometry-based viability analysis, or combinations thereof prior to culturing, organoid formation, and subsequent molecular or functional analyses. In certain embodiments, de-identified donor human milk samples are collected and bio-banked under approved protocols together with associated demographic or clinical data. In some embodiments, a minimum sample volume is obtained from each participant, and metadata associated with the donor or sample collection may include lactation stage, infant gestational age, maternal age, parity, breastfeeding frequency, date of collection, time of collection, and storage information.

For example, HM-derived cells obtained from a volunteer non-Hispanic Caucasian donor and cultured in vitro on collagen using established protocols. Here, de-identified donor human milk samples were collected, and bio-banked under approved IRB protocols. The bio-bank currently comprises at least 26 human milk samples, including 21 samples accompanied by associated demographic and/or clinical data. Inclusion criteria for volunteer participants includes: (1) lactating women, and (2) ages between 30-45. In this example, a minimum of 30 mL of milk was required from each participant. A brief questionnaire to assess eligibility for childbearing age (30-45) and lactation stage (within about 2 years) post-delivery is provided to the potential participants. In addition, the clinical questions are requested from the volunteer breast milk donors include the following: age of the infant at milk collection day, infant sex, infant weight, Gestational age at birth (term/pre-term status), maternal age, delivery method/mode (select: natural, induced, or C-section), health care access (select: Federal vs. Private), parity (number of life births and number of infants breastfeed), and frequency of breastfeeding.

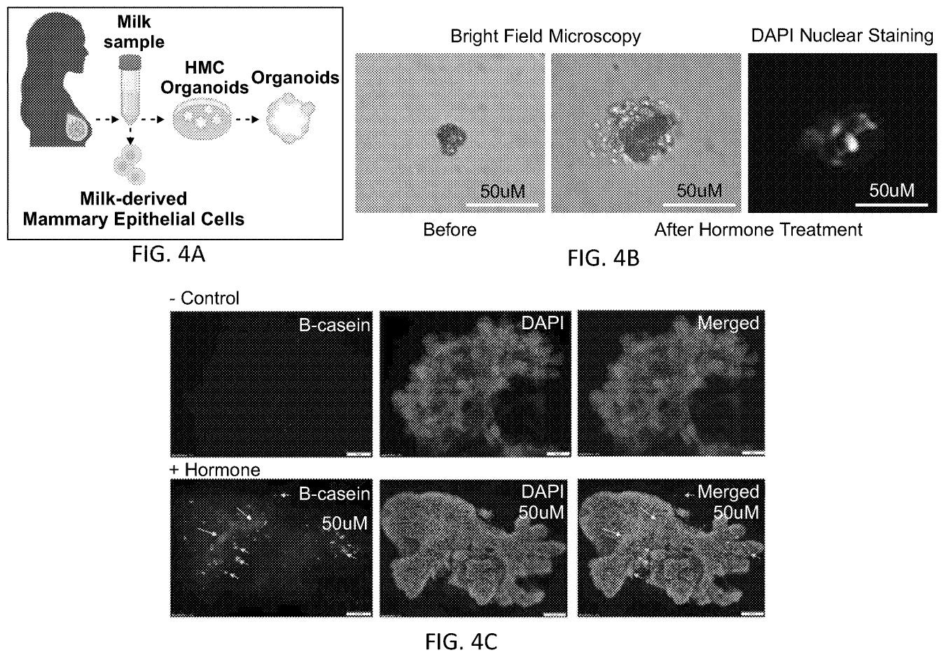

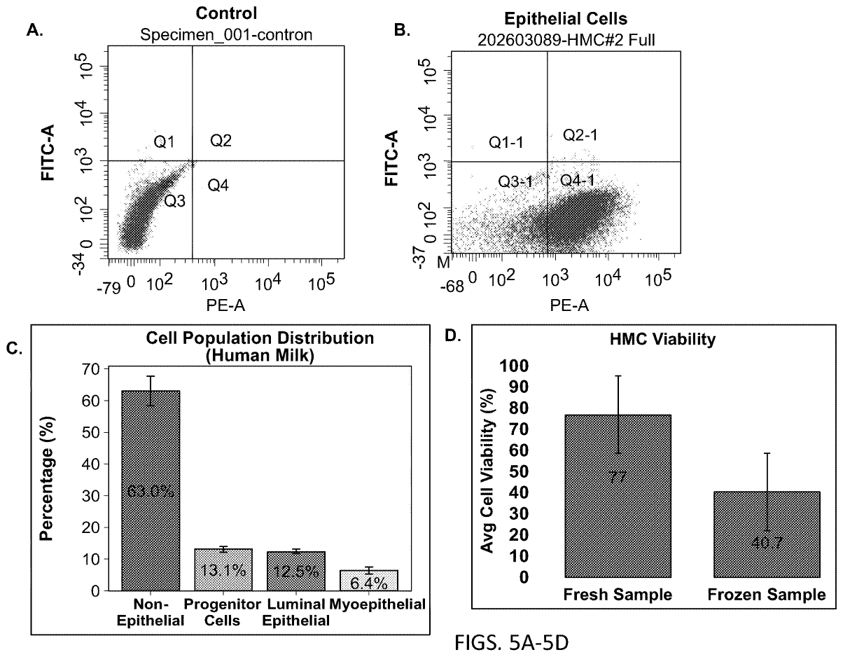

FIG. 4A is an illustration of a method of a method of generating the organoid model from human milk-derived cells. FIG. 4B in the left and middle panels present bright-field microscopy images of cultured cells before and following hormone treatment. FIG. 4B in the right panel presents fluorescence microscopy image of cultured cells following hormone treatment, showing nuclei stained with DAPI. FIG. 4C illustrates a fluorescence microscopy image of cultured cells under control conditions and following hormone treatment, showing DAPI nuclear staining. DAPI nuclear staining demonstrates the spatial distribution and orientation of cells within the human milk-derived organoids. Hormone-treated organoids exhibited increased cellular organization, density, and expanded three-dimensional morphology relative to untreated control organoids, consistent with hormone-induced proliferation and organoid maturation. The observed DAPI staining patterns further confirm successful formation and structural integrity of the organoid model following hormone stimulation. These embodiments are directed to methods of treating cultured cells with a hormone composition, and detecting treatment-associated cellular changes using both bright-field and fluorescence microscopy, including visualization of nuclear organization.

Embodiments include analysis of human milk-derived mammary epithelial cells and establishment of an ex vivo lactation model for postpartum breast cancer research. After obtaining a human milk sample from a subject, the sample may be processed immediately or cryopreserved and stored for subsequent isolation of mammary epithelial cells (MEC). The isolated cells may then be used for development of human milk-derived organoids and for downstream molecular, cellular, biomarker, and functional analyses. Cryopreservation and frozen storage do not appear to substantially reduce cellular viability, organoid-forming capacity, or downstream analytical utility relative to freshly processed samples.

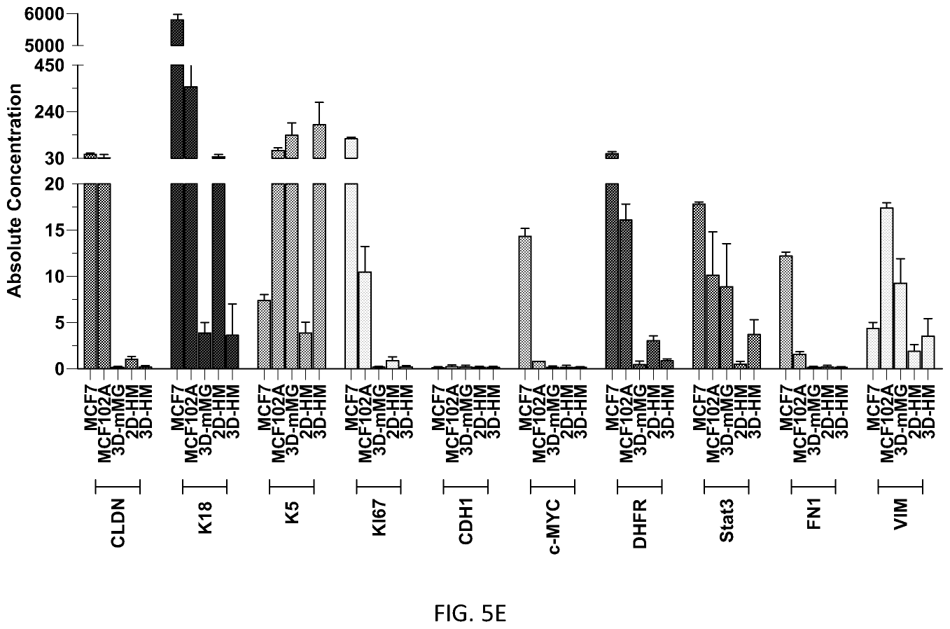

For example, milk samples from the participants are de-identified and processed immediately and/or cryopreserved and stored at approximately −80 °C for isolation of the MEC. Viability analyses as shown in FIG. 5A-5D demonstrated that cryopreservation and frozen storage of human milk samples did not significantly reduce cellular viability compared to freshly processed samples as assessed via two independent approaches: Trypan Blue exclusion assays and flow cytometry-based viability analysis. FIGS. 5A and 5B illustrate representative flow cytometry analyses (using specific epithelial, progenitor, antibodies) to identify and distinguish epithelial-associated cell populations relative to control samples. In certain embodiments, human milk samples include heterogeneous cellular populations including luminal epithelial cells, progenitor-associated cells, myoepithelial cells, and non-epithelial cell populations. FIG. 5C demonstrates the relative distribution of these cell populations within human milk-derived cellular preparations, confirming the presence of viable mammary epithelial-associated populations suitable for downstream culture and organoid generation.

FIG. 5D demonstrates comparative viability analysis of human milk-derived cells isolated from fresh versus frozen samples. In certain embodiments, fresh samples exhibit increased average cellular viability relative to frozen samples; however, frozen samples retain sufficient viable mammary epithelial cell populations for culture, molecular analysis, biobanking, and organoid generation. These findings demonstrate that frozen human milk samples remain suitable for development of mammary organoid models and breast-specific liquid biopsy applications, thereby enabling sample preservation, longitudinal studies, and scalable biobanking approaches for postpartum breast cancer (PPBC) research and breast cancer risk assessment.

FIG. 5E is a graphical representation of the expression of CLDN, K18, K5, KI67, CDH1, c-Myc, DHFR, STAT3, FN1, and VIM in five HM-derived 3D organoids. The graph demonstrates differential gene-expression profiles among mouse-derived organoids 3D mMG, human milk-derived two-dimensional cultures (2D-HM), and human milk-derived three-dimensional organoids (3D-HM), indicating that culture dimensionality, epithelial cell source, and organoid differentiation state influence molecular characteristics of the models. demonstrates that HM-derived 3D organoids exhibit a distinct gene-expression profile involving epithelial lineage markers, proliferation markers, oncogenic markers, and epithelial-to-mesenchymal transition-associated markers. Expression of K18 and CLDN1 supports preservation of mammary epithelial characterization and identity, while expression of Ki67 indicates proliferative activity within the organoid models. Differential expression of CDH1, c-MYC, STAT3, DHFR, FN1, and VIM indicates activation of breast cancer-associated, remodeling-associated, and EMT-associated pathways. Compared with 2D-HM cultures, 3D-HM organoids show molecular features consistent with a more physiologically relevant mammary epithelial model. MCF7 and MCF10A serve as comparative control cell lines for evaluating breast cancer-associated and mammary epithelial gene-expression signatures. Expression of epithelial lineage markers, including K5, K18, and CLDN, confirms preservation of mammary epithelial identity across the cultured HM-derived systems, whereas differential expression of Ki67, c-Myc, STAT3, DHFR, FN1, and VIM reflects differences in proliferation, hormone responsiveness, oncogenic signaling, epithelial-to-mesenchymal transition (EMT), and postpartum-associated remodeling and cancer susceptibility.

In an embodiment, organoids are generated using established protocols and may be subjected to pregnancy-or postpartum-mimicking hormone treatment. To generate postpartum organoids, hormone-treated organoids may be deprived of hormones to recapitulate a postpartum phase. The resulting organoids may then be used for morphological analysis, protein analysis, or both. For example, organoids from each racial background are generated in 6 days using in-house established protocols. Organoids are subjected to partum-mimicking hormones treatment for 6 days. In order to generate postpartum organoids, hormone-treated organoids are deprived of hormones for 6 days to recapitulate postpartum phase.

Embodiments include a diagnostic and research platform utilizing human milk as a breast-specific, non-invasive liquid biopsy source for assessment of breast tissue condition, breast cancer risk, and early disease detection. In some embodiments, mammary epithelial cells are isolated from human milk and cultured to develop mammary organoids. The resulting composition or organoid may be analyzed for biomarkers or gene-expression profiles associated with breast cancer susceptibility, progression, postpartum breast cancer, or treatment response, including fibronectin, E-cadherin, c-Myc, STAT3, dihydrofolate reductase, and vimentin. The disclosed systems and methods provide an accessible and cost-effective approach to breast tissue assessment, particularly for postpartum and lactating subjects.

The embodiments disclosed herein address BCa detection challenges by providing a non-invasive, accessible, and cost-effective alternative using human milk as a diagnostic medium. By isolating mammary epithelial cells from breast milk and analyzing their molecular, cellular, genetic, and/or biomarker signatures, this platform enables early detection of breast cancer risk at a molecular level. This is particularly beneficial for postpartum women, who experience increased breast cancer risk yet face a lack of screening options during this critical period. By offering accessible, cost-effective and non-invasive methods for assessing breast health, these postpartum mammary gland models (PMGM) fills a crucial gap in current breast cancer screening strategies, empowering individuals with earlier and more personalized risk assessment and potentially improving long-term health outcomes.

Human milk represents a rich source of breast-specific source of mammary epithelial cells (MECs) and, which originate directly from the lactating breast tissue. Recent research has demonstrated that the majority of epithelial cells found in human milk belong to the luminal progenitor lineage, a population known to be highly relevant in breast development and tumorigenesis. In certain embodiments, the disclosed, the PMGM embodiments exploit the presence of MECs in human milk as a unique and accessible cellular platform for studying the molecular, cellular and biomarker signature associated with breast physiology, breast cancer risk, and disease progression. Unlike traditional tissue biopsies, which require invasive procedures, human milk offers a direct and minimally invasive means of obtaining breast-derived epithelial cells. These cells retain key molecular and genetic markers reflective of the physiological and pathological states of the mammary gland, thereby enabling applications in breast cancer risk assessment, early disease detection, disease monitoring, biomarker discovery, and therapeutic response evaluation. In certain embodiments, by leveraging the natural shedding of MECs into milk, this platform enables real-time monitoring of breast health, allowing for the detection of early oncogenic changes before clinical symptoms manifest. The ability to analyze these cells for genomic, transcriptomic, and proteomic alterations provides a novel approach for identifying biomarkers associated with breast cancer initiation and progression. Additionally, as human milk is an easily obtainable biofluid from lactating mothers, this method holds significant potential for widespread clinical application, offering a patient-friendly alternative to conventional diagnostic techniques.

This platform allows for direct evaluation of viable human milk mammary epithelial cells (mMEC), providing a breast-specific sample as an extension to human milk liquid biopsies. Embodiments include new techniques to directly test the health of cancer susceptible mammary epithelial cells for early detection and biobanking for future personalized therapeutics. These embodiments have several advantages, such as optimized culturing of mMEC using unique isolation protocols to ensure the integrity of mMECs. Consistently, mMECs can be grown on natural and synthetic, 2D and 3D culturing systems, 2D-HM and 3D-HM, respectively. In addition, embodiments include methods for preservation and biological storage of isolated mMECs for future use and/or analysis. Other embodiments include a novel functional assessment platform for mMEC, specifically 2D-HM and 3D-HM. In an embodiment, mid-throughput protocols are used to test the ability of 3D-HM to form functional ex-vivo mammary glands. Furthermore, embodiments include genetic testing of 2D-HM and/or 3D-HM for high breast cancer risk (highlighted in FIG. 4). Embodiments include cultured 2D-HM and/or 3D-HM that can be evaluated with a specific gene panel associated with high pre-disposition to breast cancer. These models are ideal for young women and/or patients with inheritable pathogenic variants of breast cancer (i.e. CDH1 and/or BRAC mutations as examples).

The 2D-HM and 3D-HM models present several advantages to current detection systems. These models present non-invasive and radiation-free testing. Unlike mammography, which exposes patients to radiation, this method is completely safe for all individuals, including young lactating women who are typically underserved in breast cancer screening. These models present effective methods of detection independent of tissue density: Mammograms have reduced sensitivity in women with dense breast tissue, leading to missed diagnoses. This technology circumvents this limitation by analyzing actual mammary cells rather than imaging tissue density. These models are ideal for early detection. The American Cancer Society encourages mammograms after age 45. While only 9% of women under 45 are diagnosed annually for the disease, these younger patients exhibit more aggressive, highly metastatic forms of breast cancer that is generally “unresponsive” to standard anti-cancer therapy. These models are ideal for screening younger women before recommended mammogram screening. These models are low-cost and accessible to a broad population. Compared to expensive imaging techniques and biopsies, the methods of detection using these models are affordable and can be widely available, making early breast cancer screening more accessible to underprivileged populations. These models permit direct testing for carcinogenic cells: Current liquid biopsies of human milk only evaluate biomolecules particularly circulating tumor DNA (ctDNA). While ctDNA provides substantial knowledge of the tumor environment, its role in early cancer detection is controversial. The current mMEC technology will test the risk for and/or presence of carcinogenic transformation of the mammary cells.

Embodiments of the methods of making the organoids or 3D-HM include the following steps: obtaining a milk sample, subjecting the sample to an initial cell enrichment process, and culturing the mMECs. Embodiments of the methods include sample processing following sample collection (FIG. 4A). In an embodiment, fresh human milk samples are collected under sterile conditions to minimize contamination and degradation of cellular components. The cell pellet is washed multiple times with phosphate-buffered saline (PBS) with 2% antibiotic, such as antimycotic and 1% penicillin and streptomycin, to remove residual milk proteins and non-cellular debris. Methods can include the step of initial cell enrichment. Milk is subjected to 3-5 low-speed centrifugation (~500 ×g for 10 minutes at 4 °C) to separate fat globules, which are discarded. Isolated pellets include primarily mMECs (specifically alveolar, myoepithelial, and luminal stem/progenitor cells), adipocytes, and circulating immune cells. Methods can include a quality control process. One such QC process includes fluorescence-activated cell sorting (FACS). In an embodiment, to obtain a pure mMEC population, epithelial-specific markers such as EpCAM and cytokeratin 8/18 are used for sorting. Negative selection markers (e.g., CD45, CD14) may be incorporated to deplete immune cells.

Methods can include a cell culturing process or 2D-HM. Additional cells are cultured in optimum adherent conditions to promote proliferation of adherent mMECs and eliminate residual cells (i.e. adipocytes and immune cells). Specifically, human mMECs are maintained in epithelial-specific mMEC growth media supplemented with essential growth factors such as FGF2, EGF, Rock inhibitor Y-27632, Forskolin, Insulin, Heparin, Cholera toxin.

Methods can include a live-cell bio-biobanking process (e.g., as shown in FIG. 5D). Following FACS isolation, purified mMECs are assessed for viability using Trypan Blue exclusion assay. Viable cells are cryopreserved in fetal bovine serum (FBS) with 10% dimethyl sulfoxide (DMSO) or other optimized cryoprotectant solutions, ensuring maximum post-thaw recovery. Slow freezing is performed using a controlled-rate freezing system or by placing vials in a ThermoFisher Scientific Mr. Frosty container at −80 °C overnight before transferring them to liquid nitrogen (−196 °C) for long-term storage.

Embodiments include scalable and physiologically relevant platforms for studying mMEC phenotypic behavior, detecting early molecular oncogenic reprogramming, and assessing functional abnormalities in mMECs. In certain embodiments, breast-associated abnormalities include benign proliferative changes, atypical ductal or lobular hyperplasia, ductal carcinoma in situ (DCIS), lobular carcinoma in situ (LCIS), hormone receptor-positive or -negative lesions, HER2-amplified lesions, high-proliferation phenotypes marked by elevated biomarkers, and invasive ductal or lobular carcinomas.

Methods include a 2D culture system for carcinogenic growth. In an embodiment, human mMECs are seeded onto 6-well plates-coated with collagen and maintained in epithelial-specific mMEC growth media (FIGS. 4B-4C). mMEC monolayer culture system allows for assessment of cell proliferation, morphological potential studies analysis using real-time imaging, viability assays, adhesion, spreading, and colony formation.

Embodiments can include molecular analysis using a gene panel as shown for FIG. 5E. To assess molecular characteristics of cultured mammary epithelial cell preparations and mammary organoids, RNA may be isolated and analyzed by digital droplet polymerase chain reaction to quantify target transcripts. In certain embodiments, the gene panel includes two or more biomarkers selected from Ki67, keratin 5, keratin 18, E-cadherin, claudin, STAT3, vimentin, dihydrofolate reductase, c-Myc, and fibronectin. In an example, to assess the genetic predisposition and molecular characteristics of 2D-HM and 3D-HM models, RNA isolation, ddPCR analysis of transcript copy numbers, and primer design for the custom gene panel are conducted. High-quality genomic RNA are extracted from purified 2D-HM and 3D-HM using standard nucleic acid isolation kits and subsequent quantification of target gene transcripts. Specifically, the gene panel includes experimentally validated list of aberrantly expressed gene transcripts associated with inheritable predisposition to breast cancer, PPBC gene-drivers, and breast cancer proto-oncogenes as defined by us and others in the field (Hu et al., 2021).

Embodiments can include functional mammary epithelial cell analysis using the mouse models shown in FIGS. 1A, 1B, 2A-2I, and 3A-3I and the human model shown in FIGS. 4B-4C. For the human model, mammary epithelial cells may be cultured within extracellular matrix-rich hydrogels, such as collagen, laminin, heparan sulfate proteoglycans, entactin/nidogen, growth factors, or a combination thereof, to promote formation of mammary organoids. In one embodiment, Corning® Matrigel® matrix is used. This three-dimensional culture approach supports differentiation, polarity, and functional modeling of mammary tissue. Following isolation and enrichment of mammary epithelial cells, the cells may be counted and embedded in a hydrogel matrix. After hydrogel polymerization, an organoid culture medium supplemented with growth factors may be added, and the culture may be maintained under standard conditions with regular media replenishment to support organoid growth and differentiation. In certain embodiments, a lactation hormone cocktail is administered after organoid establishment, and the organoids are subsequently evaluated for hormone-induced differentiation, morphological remodeling, or functional maturation.

This advanced lactation model provides a powerful platform for studying mammary gland biology, lactogenic function, and oncogenic transformation in a physiologically relevant setting. In certain embodiments, the model is used to quantify morphological and molecular changes associated with lactation, involution, postpartum remodeling, and/or oncogenesis. Such analysis may use one or more measured inputs from the disclosed assays, including biomarker expression values, transcript copy numbers, image-derived morphology features, viability metrics, or combinations thereof, to identify whether a test sample differs from a reference sample or profile associated with breast cancer, postpartum remodeling, treatment response, or disease progression. In certain embodiments, the measured inputs are compared to a reference population, a non-cancer control, a postpartum control, a parity-matched control, a previous sample from the same subject, or combinations thereof.

In certain embodiments, measuring one or more biomarkers associated with breast cancer includes nucleic acid analysis of the human milk-derived mammary epithelial cells, the cultured mammary epithelial cell preparation, and/or the mammary organoid. Suitable nucleic acid analyses include reverse transcription polymerase chain reaction, quantitative polymerase chain reaction, digital droplet polymerase chain reaction, targeted sequencing, RNA sequencing, or combinations thereof. In some embodiments, digital droplet polymerase chain reaction is performed on a gene panel comprising at least two, at least three, at least four, or more of Ki67, K5, K18, CDH1, CLDN, STAT3, VIM, DHFR, c-Myc, FN1, and combinations thereof. In certain embodiments, absolute or normalized copy number, expression level, fold-change, presence, or absence of one or more genes in the panel is compared to a reference profile obtained from a non-cancer control, a postpartum control, a parity-matched control, an age-matched control, or a prior sample from the same subject.

In certain embodiments, measuring one or more biomarkers associated with breast cancer additionally or alternatively includes microscopy-based image analysis of the mammary organoid or cultured mammary epithelial cell preparation. Brightfield microscopy, fluorescence microscopy, confocal microscopy, immunofluorescence imaging, or combinations thereof may be used to acquire images for analysis. Quantified morphology features may include organoid size, area, perimeter, branching number, branch length, circularity, lumen formation, lumen diameter, wall thickness, polarity, cell density, and/or marker localization. In some embodiments, the quantified morphology features are compiled into a control morphology profile and compared with a morphology profile from a test sample to identify postpartum remodeling, involution-like changes, oncogenic transformation, treatment response, or disease progression.

In certain embodiments, the disclosed methods are performed longitudinally using a first human milk sample and a second human milk sample obtained from the same subject at different times, and optionally additional samples collected over time. The samples may be collected before treatment, during treatment, after treatment, during lactation, during involution, during the postpartum period, or combinations thereof. In some embodiments, a biomarker level, biomarker pattern, transcript copy number, or morphology feature from the first sample is compared with a corresponding result from the second sample to monitor treatment response, detect disease progression, assess recurrence risk, or evaluate temporal changes in breast tissue condition.

In certain embodiments, the disclosed analysis uses a single biomarker measurement, multiple biomarker measurements, one or more morphology features, or a combination of molecular and morphology features. The resulting analysis may be stored, displayed, or reported and may be used to guide additional testing, monitoring, or therapeutic decision-making.

Human milk is readily accessible and provides information regarding the condition of lactating breast tissue. Human milk contains predominantly viable epithelial cells from the lumen of the mammary gland, including luminal progenitor cells. Accordingly, organoids can be generated from human milk-derived mammary epithelial cells. In certain embodiments, a postpartum mammary gland model is established from isolated mouse mammary epithelial cells and from human milk-derived mammary epithelial cells. High parity correlates with greater risk of PPBC.

In certain embodiments, the disclosed PMGM models provide platforms for evaluation of PPBC-associated physical, molecular, cellular, and biomarker signatures for use in breast cancer risk assessment and early detection. In some embodiments, the disclosed systems and methods provide accessible and cost-effective approaches for breast health assessment and PPBC risk stratification relative to conventional imaging-based screening methods. In certain embodiments, a PMGM is generated from human milk-derived cells to assess PPBC risk in a subject. Such approaches may be particularly useful in underserved settings in which access to imaging-based screening is limited. In further embodiments, the disclosed systems and methods enable minimally invasive analysis of breast-specific cellular and molecular characteristics associated with PPBC susceptibility, progression, and therapeutic response.

Epidemiological studies indicate that women exhibiting a higher incidence of aggressive breast cancer frequently also exhibit higher parity and increased postpartum breast cancer risk. The basis for the increased susceptibility to PPBC remains incompletely understood. Accordingly, embodiments disclosed herein provide platforms and models for investigating molecular, cellular, hormonal, and/or involution-associated mechanisms involved in postpartum mammary gland remodeling and their association with PPBC initiation, progression, and risk. In certain embodiments, the disclosed systems and methods enable evaluation of differentiation states, involution-associated remodeling, biomarker expression, and functional changes occurring within postpartum breast tissue and mammary organoid models following pregnancy-associated and/or parity-associated hormonal exposure.

Human milk (HM) contains a broad range of bioactive components. Studies indicate that HM also contains viable cells of the epithelial luminal progenitor lineage. Such cells are secreted into milk during lactation and therefore provide a source for evaluating functional mammary gland changes during lactation and the postpartum period. In certain embodiments, HM-derived cells are used to identify molecular, cellular, or biomarker features associated with future breast cancer development, thereby contributing to development of a preclinical tool for early breast cancer detection. The disclosed systems and methods thereby provide minimally invasive platforms for breast health assessment, biomarker discovery, early disease detection, and personalized risk stratification.

In some embodiments, breast milk-derived cells are utilized to generate a three-dimensional mammary organoid model (3D-HM). Such ex vivo organoid models may recapitulate structural, functional, and molecular characteristics of mammary tissue and provide platforms for investigation of mammary gland biology, postpartum involution, tumorigenesis, hormonal responsiveness, and breast cancer-associated pathways. Despite advances in organoid technologies, there remains a need for physiologically relevant postpartum mammary organoid models capable of recapitulating lactation-associated and involution-associated processes across diverse patient populations, including populations having distinct racial, ethnic, genetic, and parity-associated backgrounds. In certain embodiments, the disclosed systems and methods provide cost-effective and accessible models utilizing HM-derived cells as prognostic tools for evaluating PPBC susceptibility, parity-associated carcinogenesis, biomarker expression, and therapeutic response in high-risk and postpartum populations. In certain embodiments, the disclosed models further provide diagnostic, prognostic, therapeutic, and research platforms for evaluating gene-defined PPBC and mechanisms associated with parity-driven breast cancer initiation and progression.

Accordingly, development of new research models is useful for studying PPBC development and initiation of carcinogenesis across different populations. In certain embodiments, a postpartum organoid model using HM-derived cells from women of different ages and parity levels is used to study PPBC susceptibility. In some embodiments, the disclosed postpartum organoid models enable investigation of relationships among age, parity, hormonal exposure, involution-associated remodeling, molecular signatures, and susceptibility to PPBC initiation and progression. The disclosed models further provide platforms for biomarker analysis, personalized risk assessment, therapeutic response evaluation, and investigation of parity-associated and postpartum-associated mechanisms contributing to breast carcinogenesis.

Pregnancy, lactation, and the postpartum period induce substantial developmental changes in the mammary gland (MG). These phases are considered windows of breast cancer risk. During lactation, the MG expands (for example, 10-fold) to generate secretory cells. Following cessation of lactation, in a process referred to as involution, the MG undergoes extensive clearance and apoptosis process to remove approximately 80% of secretory epithelial cells. Studies indicate that postpartum MG involution resembles a wound-healing process and has been associated with tumor-promotional conditions. However, the manner in which healthy epithelial cells may transition into cancer cells during involution and the postpartum years remains incompletely understood. Further, research indicates that factors including higher parity and older maternal age at childbirth are associated with increased PPBC risk, which may persist for extended periods, for example two decades, following childbirth. SEER-based studies have also indicated that African American and Hispanic women often present with more aggressive forms of breast cancer. Such groups may also exhibit higher parity. However, the relationship between parity within such populations and increased susceptibility to PPBC remains to be determined.

In certain embodiments, the PMGM is generated to reflect pregnancy-associated, lactation-associated, and/or involution-associated changes in mammary epithelial biology. In certain embodiments, the methods include enrichment of mammary epithelial cells (MECs) from female nulliparous and parous animals. The MECs may be embedded in a Matrigel® matrix to support reconstitution of three-dimensional organoids to resemble a “gland on a plate” capable of recapitulating structural and functional characteristics of mammary gland tissue. To model a postpartum phase of mammary gland development (a “gland on a plate”), mouse mammary organoids may be exposed to pregnancy-associated and lactation-associated hormones followed by hormone withdrawal (FIGS. 2A-2H and FIGS. 3A-3H). Studies indicate that pregnancy/lactation hormone-treated nulliparous organoids undergo a morphological transformation characterized by secretory branching patterns (FIG. 2B), similar to parous hormone-treated organoids (FIG. 3F), relative to corresponding untreated control organoids (FIG. 2B and FIG. 3B). Additionally, when subjected to hormone withdrawal, nulliparous organoids exhibit postpartum morphology characterized by reduced size and branching (FIGS. 2G-2H). In contrast, postpartum organoid models derived from parous mammary glands having two to three prior pregnancies do not exhibit the same degree of change in branching following hormone deprivation (FIGS. 3G-3H). These findings indicate parity-associated differences in postpartum remodeling dynamics and provide physiologically relevant platforms for evaluating mechanisms associated with postpartum breast cancer susceptibility, involution-associated carcinogenesis, and parity-associated breast cancer risk.

In some embodiments, the postpartum organoid model mimics morphological aspects of the mammary gland in an in vivo-like environment. Human milk contains numerous non-nutritional bioactive components that may provide information regarding the condition of the lactating breast and may be used as potential biomarkers for early detection of PPBC. In certain embodiments, viable HM-derived cells are isolated from a donor sample and cultured in vitro on collagen using established protocols (FIG. 3A and FIGS. 4A-4B).

In certain embodiments, human milk (HM) samples are obtained from subjects representing diverse racial, ethnic, parity, age, and genetic backgrounds to generate human postpartum mammary gland models (PMGM). In some embodiments, such samples are utilized for generation of breast-specific organoid platforms for evaluation of postpartum breast cancer (PPBC) susceptibility, biomarker expression, hormonal responsiveness, and involution-associated remodeling across distinct patient populations.

In certain embodiments, HM samples obtained from subjects representing diverse racial and ethnic backgrounds are utilized for generation of mammary organoids. In some embodiments, organoids are generated within approximately six days through isolation and culture of mammary epithelial cells (MECs) derived from HM samples. In certain embodiments, the organoids are subjected to partum-mimicking hormone treatment that stimulates expansion of organoid size and emergence of branch-like extensions from the organoid core. Such expansion may be analogous to epithelial expansion and enhanced ductal formation observed during pregnancy and postpartum lactation. In certain embodiments, branching is not observed, or is substantially reduced, in no-hormone-treated control organoids. To generate postpartum organoids, hormone-treated organoids may be deprived of hormones for about six days to recapitulate a postpartum phase. HMC-derived and MEC-derived organoids from each developmental stage, including pre-treatment, hormonally stimulated, and postpartum stages, may be collected for characterization studies. In certain embodiments, proper organoid development is evaluated based on bilayer luminal and myoepithelial composition, including analysis of keratin 8 and keratin 18 markers. Studies further indicate that organoids may produce milk protein β-casein in response to parity-mimicking hormone treatment. Accordingly, the organoids may be evaluated for responsiveness to a lactation hormone cocktail to assess hormone-induced differentiation, morphological remodeling, and functional maturation. In addition, a Ki67 proliferation marker may be used to evaluate proliferation and growth of the organoids in the presence and absence of hormones. Postpartum organoid size and morphology, including branch number, may also be examined.

Following lactation, the mammary gland (MG) undergoes a restructuring process referred to as involution, during which the MG returns toward a pre-pregnancy state. In certain embodiments, involution is modeled in the PMGM by removal of pregnancy-associated and lactation-associated hormones. In the absence of parity-mimicking hormonal stimulation, organoids may undergo an involution-like postpartum process characterized by increased apoptosis, reduced proliferation, reduced organoid size, and reduced branching, as demonstrated in the mouse PMGM (FIGS. 4B and 4C). HMC-derived and MEC-derived organoid changes from each background, including changes in size, shape, and branching, may be analyzed and compared with a no-hormone-treated control group. In certain embodiments, organoid size and morphological characteristics are quantified using ImageJ software and associated analytical tools. In further embodiments, changes in PPBC-associated genes, including fibronectin, inheritable breast cancer-associated genes including CDH1 and c-Myc, proto-oncogenes including STAT3 and DHFR, and epithelial-to-mesenchymal transition markers including vimentin, are evaluated as biomarkers that may be altered in breast cancer models. In certain embodiments, such biomarker patterns may indicate differential susceptibility to PPBC across subject populations. Where DNA damage response dysregulation is identified, targeted therapeutic approaches, including PARP inhibitor-based approaches, may be evaluated in appropriate subject populations, such as African American women.

High parity is associated with greater risk of PPBC. Accordingly, embodiments disclosed herein include parity-mimicking and multiparity-mimicking hormonal stimulation models for evaluation of postpartum-associated molecular and morphological changes in mammary epithelial cell (MEC)-derived organoids. In certain embodiments, mammary glands from 24-week-old nulliparous female mice and 24-week-old parous female mice having two to three prior pregnancies are utilized to generate organoids. Following organoid establishment for about six days, organoids from both the nulliparous and parous groups may be treated with or without parity-mimicking hormonal stimulation for about six days. In certain embodiments, both nulliparous and parous organoids exhibit expansion, branching, secretory differentiation, and morphological remodeling, whereas untreated organoids exhibit minimal morphological change. Following withdrawal of hormone treatment for about six days, the organoids may be evaluated for involution-associated changes, including alterations in organoid size, branching architecture, structural regression, biomarker expression, and functional remodeling. In some embodiments, nulliparous postpartum organoids exhibit involution-associated reduction in organoid core size and branching patterns consistent with previously developed PMGM models. In contrast, parous or multiparous MEC-derived organoids may exhibit distinct involution dynamics, remodeling responses, and molecular signatures associated with prior pregnancy exposure and multiparity history. In certain embodiments, multiparity may be associated with reduced involution and increased branching relative to parous no-hormone controls. Analysis of hormonal response and involution may be conducted as described herein and corresponding drawings (FIGS. 2A-2H and FIGS. 3A-3H).

Analogous to the PMGMs disclosed here, multiparity-mimicking postpartum organoids can be cultivated and evaluated for developmental changes. Accordingly, in certain embodiments, an animal-derived PMGM model provides a platform for evaluating relationships between selected epidemiological factors, including parity and multiparity history, and PPBC-associated molecular, cellular, and functional alterations, as well as PPBC onset.

Additional objects, features and advantages of the present disclosure will become apparent from the foregoing drawings, detailed description, and examples. It should be understood, however, that the drawings, detailed description, and examples, while indicating specific embodiments of the disclosure, are given by way of illustration only and are not meant to be limiting. Additionally, it is contemplated that changes and modifications within the spirit and scope of the disclosure will become apparent to those skilled in the art from the detailed description. In further embodiments, features from specific embodiments may be combined with features from other embodiments. For example, features from one embodiment may be combined with features from any of the other embodiments. In further embodiments, additional features may be added to the specific embodiments described herein.

What is claimed is:

1. A breast-derived cellular composition comprising mammary epithelial cells obtained from a human milk sample, wherein the mammary epithelial cells are capable of being cultured under organoid-forming conditions to produce a mammary organoid.

2. The composition of claim 1, wherein the mammary epithelial cells are disposed in a three-dimensional matrix.

3. The composition of claim 2, wherein the three-dimensional matrix comprises collagen, laminin, heparan sulfate proteoglycans, entactin/nidogen, growth factors, or a combination thereof.

4. The composition of claim 1, wherein the mammary organoid exhibits secretory differentiation, branching morphology, milk secretion, or a combination thereof.

5. The composition of claim 1, wherein the composition or the mammary organoid produced therefrom is evaluated for one or more biomarkers associated with proliferation, epithelial differentiation, stemness, oncogenic transformation, or epithelial-to-mesenchymal transition.

6. The composition of claim 5, wherein the one or more biomarkers comprise Ki67, keratin 5, keratin 18, E-cadherin, claudin, STAT3, vimentin, dihydrofolate reductase, c-Myc, fibronectin, or a combination thereof.

7. The composition of claim 1, wherein the human milk sample is enriched for mammary epithelial cells prior to culturing.

8. The composition of claim 7, wherein enriching comprises centrifugation, fluorescence-activated cell sorting, marker-based selection, negative immune-cell depletion, or a combination thereof.

9. A method of producing a breast-derived cellular composition or a mammary organoid, the method comprising:
obtaining a human milk sample;
processing the human milk sample to provide mammary epithelial cells; and
culturing the mammary epithelial cells under conditions sufficient to generate the breast-derived cellular composition or the mammary organoid.

10. The method of claim 9, further comprising cryopreserving the human milk sample or the mammary epithelial cells prior to the culturing step.

11. The method of claim 9, wherein the processing step comprises washing, centrifuging, enriching, sorting, thawing, or a combination thereof.

12. The method of claim 9, wherein the culturing step comprises culturing the mammary epithelial cells in a three-dimensional matrix.

13. The method of claim 9, further comprising exposing the mammary organoid to one or more pregnancy-associated hormones, lactation-associated hormones, or a combination thereof.

14. The method of claim 13, further comprising withdrawing at least one of the hormones to model an involution or postpartum state.

15. A method of assessing a condition of breast tissue in a subject, the method comprising:
obtaining a human milk sample from the subject;
isolating or enriching mammary epithelial cells from the human milk sample;
culturing the mammary epithelial cells to produce a mammary organoid or a cultured mammary epithelial cell preparation;
measuring, in the mammary organoid or the cultured mammary epithelial cell preparation, a level or pattern of one or more biomarkers associated with breast cancer; and
comparing the measured level or pattern to a reference profile and identifying the subject as having, or as being at risk of having, a breast-associated abnormality based on the comparison.

16. The method of claim 15, wherein the breast-associated abnormality comprises postpartum breast tissue abnormality.

17. The method of claim 15, wherein comparing the measured level or pattern to a reference profile comprises comparing the measured level or pattern to a reference profile obtained from a healthy control, a postpartum control, or a prior sample from the subject.

18. The method of claim 15, wherein the measuring of the level of the one or more biomarkers associated with breast cancer comprises digital droplet polymerase chain reaction performed on a gene panel comprising at least two of Ki67, keratin 5, keratin 18, E-cadherin, claudin, STAT3, vimentin, DHFR, c-Myc, fibronectin, or a combination thereof.

19. The method of claim 15, wherein the measuring of the pattern of the one or more biomarkers associated with breast cancer comprises microscopy-based image analysis of the mammary organoid to quantify size, branching, circularity, lumen formation, or a combination thereof, and wherein the comparing comprises comparing the quantified morphology to a control morphology profile.

20. The method of claim 15, wherein the method is performed on a first human milk sample and a second human milk sample obtained from the subject at different times, and wherein the step of comparing comprises comparing a biomarker level or morphological feature from the first sample to a corresponding biomarker level or morphological feature from the second sample to monitor treatment response or disease progression.
